(19) United States
(12) Patent Application Publication
(10) Pub. No.: US 2016/0270728 A1
(43) Pub. Date: Sep. 22, 2016

(54) GARMENT WITH SAFETY FEATURES

(71) Applicant: TELEPORT L.A. CORPORATION, Calgary (CA)

(72) Inventors: Sean Duane McGinnis, Calgary (CA); Paul Espino Gonzalez, Los Angeles, CA (US); Jeffrey Ryan Enerson, Calgary (CA)

(21) Appl. No.: 15/035,140

(22) PCT Filed: Nov. 7, 2014
(86) PCT No.: PCT/IB2014/003081

§ 371 Date: May 6, 2016

Related U.S. Application Data
(60) Provisional application No. 61/902,074, filed on Nov. 8, 2013.
(60) Provisional application No. 62/009,048, filed on Jun. 6, 2014.

(51) Int. Cl. A61B 5/00; A62B 17/00; A61B 5/0205; A41D 1/00; A41D 31/00; A41D 13/00; A41F 9/02

(52) U.S. Cl. CPC A61B 5/6804; A41D 13/0007; A62B 17/006; A41F 9/025; A41D 1/002; A41D 31/0027; A61B 5/0205; A61B 5/021

(57) ABSTRACT

Safety garments for use in hazardous environments are disclosed. An exemplary safety garment comprises: a first protective covering configured to cover at least part of a torso of a wearer; a second protective covering configured to cover at least part of a limb of the wearer; and a flexible panel interconnecting the first protective covering to the second protective covering. The flexible panel facilitates movement of the limb of the wearer relative to the torso of the wearer by accommodating relative movement between the first protective covering and the second protective covering. Also disclosed are a safety garment comprising a wearer-carrying harness, a safety garment comprising a refillable heat extraction pack for cooling the wearer and a safety garment comprising a deployable shield for protecting the wearer upon detection of an event.

### CROSS REFERENCE TO RELATED APPLICATIONS AND CLAIMS OF PRIORITY

The present application claims priority to U.S. provisional patent application No. 62/009,048 filed on Jun. 6, 2014 the entire contents of which are hereby incorporated by reference.

The present application also claims priority to U.S. provisional patent application No. 61/902,074 filed on Nov. 8, 2013 the entire contents of which are hereby incorporated by reference.

### TECHNICAL FIELD

The disclosure relates generally to garments, and more particularly to safety garments incorporating features enabling the use of the garment in a variety of applications, including the oil and gas field, hazardous and other environments.

### BACKGROUND OF THE ART

Many individuals are subjected to a variety of environmental dangers and hazardous environments during the course of their employment, for example those in the oil and gas industry, firefighters, lumberjacks, on-site engineers, and construction workers. This may include the risk of fire, heat, hot steam, exposure to harmful or toxic chemicals including certain liquid substances, for example oil, fumes or gases.

These potential dangers pose significant risk of injury to these individuals as they carry out specific tasks associated with their employment. These tasks necessitate the use of tools or other equipment by the individuals.

There is therefore a need for an improvement in safety garments for use by individuals working in hazardous environments.

### SUMMARY

The disclosure describes garments configured to protect a user from one or more environmental dangers, such as those encountered during oil and gas field exploration.

In one aspect, the disclosure describes a garment for use in hazardous or other environments, the garment comprising: a first covering configured to cover at least part of a torso of a wearer; a second covering configured to cover at least part of a limb of the wearer; and a first flexible panel extending from the first covering to the second covering and interconnecting the first covering to the second covering, the first flexible panel completely surrounding a limb passage adapted to receive the limb of the wearer, the first flexible panel facilitating movement of the limb of the wearer relative to the torso of the wearer by accommodating relative movement between the first covering and the second covering.

In another aspect, the disclosure describes a safety garment for use in hazardous environments, the safety garment comprising: a covering configured to cover at least part of a torso of a wearer of the garment; and a wearer-carrying harness integrated into the covering, the wearer-carrying harness being configured to support a weight of the wearer and permit carrying of the wearer, the harness comprising: a pair of shoulder straps, each of the shoulder straps being configured to completely encircle a respective shoulder of the wearer; and a grasping device attached to the pair of shoulder straps, the grasping device being accessible from outside of the covering, the shoulder straps and the grasping device together defining a load path to permit carrying of the wearer.

In another aspect, the disclosure describes a safety garment for use in hazardous environments. The safety garment comprises a covering configured to cover at least part of a torso of a wearer of the garment; and a wearer-carrying harness integrated into the covering, the wearer-carrying harness being configured to support a weight of the wearer and permit carrying of the wearer, the harness comprising: one or more lower limb straps, each of the one or more lower limb straps being configured to completely encircle a respective lower limb of the wearer; and a grasping device attached to the one or more lower limb straps, the grasping device being accessible from outside of the covering, the one or more lower limb straps and the grasping device together defining a load path to permit carrying of the wearer.

In yet another aspect, the disclosure describes a garment for use in hazardous or other environments. The garment comprises a covering configured to cover at least part of a wearer of the garment; a refillable heat extraction pack attached to the covering, the pack defining a chamber for receiving and releasably retaining a cooling medium therein for absorbing heat from the wearer; and a flow control device for permitting the release of the cooling medium from the chamber via a port after a cooling capacity of the cooling medium has been reduced.

In another aspect, the disclosure describes a safety garment for use in hazardous environments. The safety garment comprises: a covering configured to cover at least part of a wearer of the garment; a sensor for detecting an event associated with the wearer; a deployable shield attached to the covering; an actuator operatively coupled to the sensor and to the deployable shield, the actuator being configured to cause deployment of the deployable shield upon detection of the event by the sensor.

In another aspect, the disclosure describes a garment. The garment comprises: a covering configured to cover at least a portion of a wearer, the covering extending between a first location and a second location spaced apart from the first location; and a flexible panel, the covering overlaying the flexible panel, the flexible panel being attached to the covering at the first location and the second location; wherein the covering comprises excess material between the first location and the second location, the excess material being configured to accommodate a stretching of the flexible panel.

Further details of these and other aspects of the subject matter of this application will be apparent from the detailed description and drawings included below.

### DESCRIPTION OF THE DRAWINGS

Reference is now made to the accompanying drawings, in which:

FIG. 1A shows an axonometric view of an exemplary garment;

FIGS. 1B and 1C show side elevation views of an exemplary garment comprising a flexible panel;

FIG. 1D shows a rear elevation view of another exemplary garment comprising a flexible panel;

FIG. 1E shows a side elevation view of an exemplary garment comprising a flexible panel;

FIG. 1F shows a front elevation view of another exemplary garment comprising a flexible panel;

FIG. 1G shows a front elevation view of an exemplary garment comprising a flexible panel;

FIG. 1H shows a rear elevation view of an exemplary garment comprising flexible panels;

FIG. 1I shows a side elevation view of an exemplary garment comprising flexible panels;

FIG. 1J shows a top elevation view of an exemplary garment comprising flexible panels;

FIG. 1K shows a bottom elevation view of an exemplary garment comprising flexible panels;

FIG. 2A shows an axonometric view of another exemplary garment comprising a wearer-carrying harness;

FIG. 2B shows a top elevation view of an exemplary garment comprising a wearer-carrying harness further comprising a flap;

FIG. 2C shows a rear elevation view of an exemplary garment of the present disclosure further comprising a wearer-carrying harness;

FIG. 2D shows an axonometrc view of an exemplary garment comprising a wearer-carrying harness;

FIG. 2E shows an axonometric view of an exemplary garment comprising a wearer carrying harness;

FIG. 3A shows a schematic view of an exemplary garment comprising a heat extraction pack;

FIG. 3B shows a front elevation view of an exemplary garment comprising a heat extraction pack;

FIG. 3C shows a front elevation view of another exemplary garment;

FIG. 3D shows a front elevation view of another exemplary garment;

FIG. 4 shows an axonometric view of another exemplary safety garment comprising a deployable shield;

FIG. 5A shows a cross sectional view of another exemplary safety garment comprising a flexible panel in a non-stretched configuration;

FIG. 5B shows a cross sectional view of the garment of FIG. 5A where the flexible panel is in a stretched configuration;

FIG. 6 shows an axonometric view of an exemplary garment comprising a flexible panel;

FIG. 7 shows an axonometric view of an exemplary garment comprising a flexible panel;

FIG. 8 shows an axonometric view of an exemplary garment comprising a flexible panel;

FIG. 9 shows a front elevation view of an exemplary garment comprising a flexible panel;

FIG. 10 shows a rear elevation view of an exemplary garment comprising a flexible panel;

FIG. 11 shows a rear elevation view of an exemplary garment in a reversed configuration having a flexible panel;

FIG. 12 shows a front elevation view of an exemplary garment in a reversed configuration comprising a flexible panel;

FIG. 13 shows an axonometric view of an exemplary garment comprising a pleat;

FIG. 14 shows an axonometric view of an exemplary garment comprising a pleat;

FIG. 15 shows a rear elevation view of an exemplary garment comprising a pleat;

FIG. 16 shows a rear elevation view of an exemplary garment;

FIG. 17 shows a rear elevation view of an exemplary garment;

FIG. 18 shows a side elevation view of an exemplary garment comprising a pocket;

FIG. 19 shows an axonometric view of an exemplary garment comprising pockets;

FIG. 20 shows side elevation view of an exemplary garment comprising pockets;

FIG. 21 shows a side elevation view of an exemplary garment comprising pockets;

FIG. 22 shows a side elevation view of an exemplary garment comprising pockets;

FIG. 23 shows front and rear elevation views of an exemplary garment;

FIG. 24 shows front and rear elevation views of an exemplary safety garment;

FIG. 25 shows front and rear elevation views of an exemplary safety garment.

### DETAILED DESCRIPTION

Aspects of various embodiments are described through reference to the drawings.

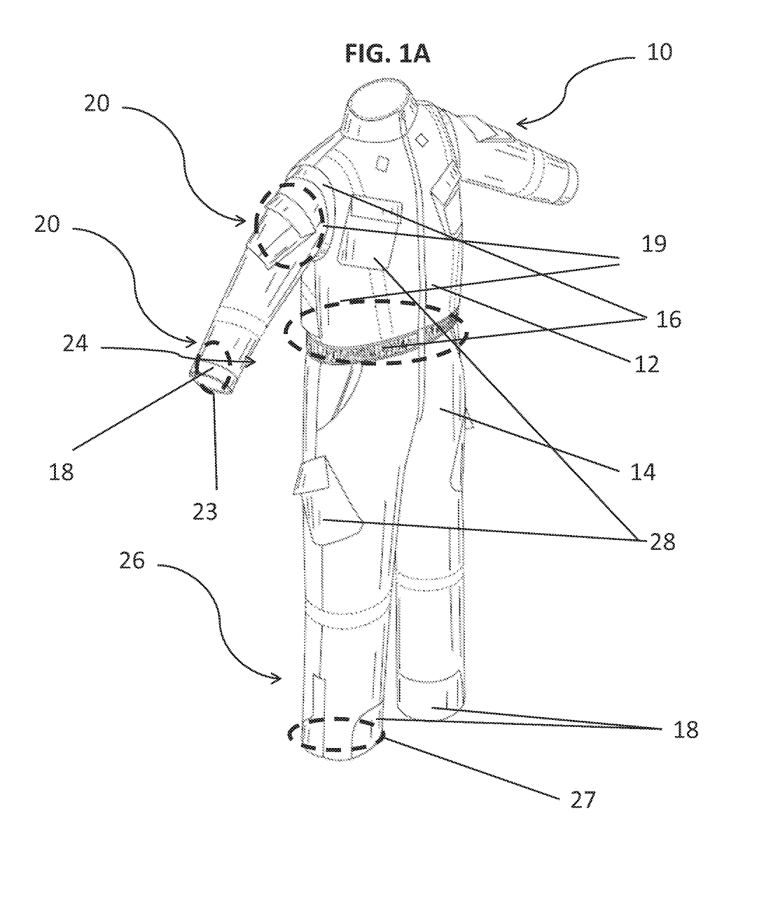

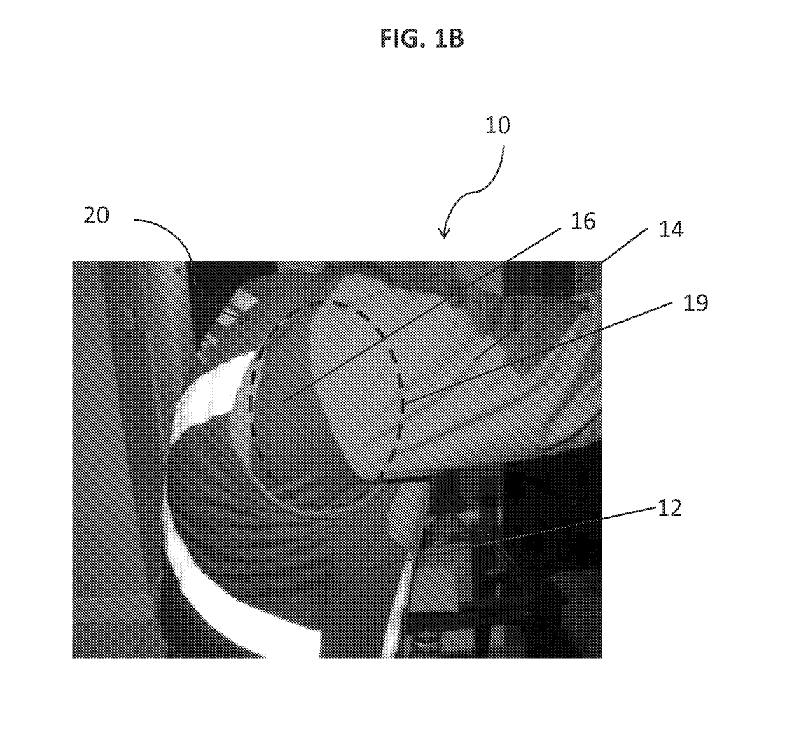

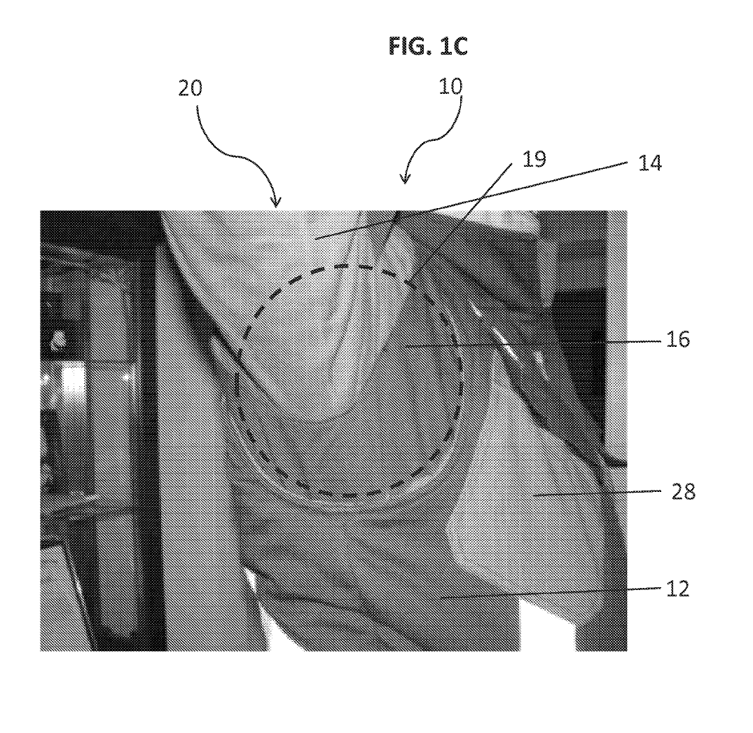

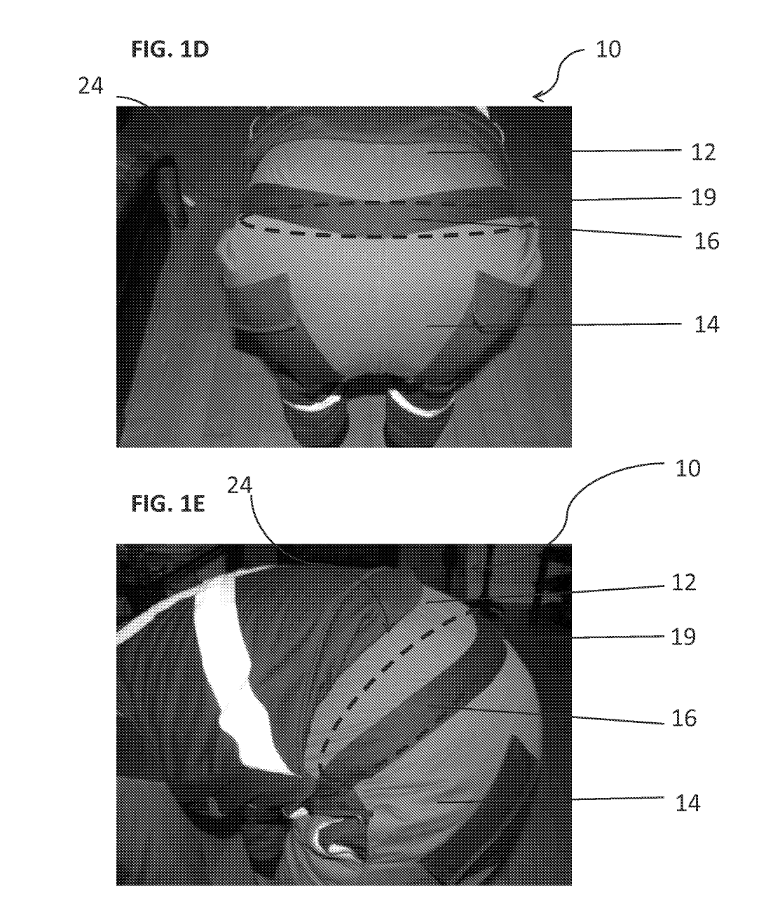

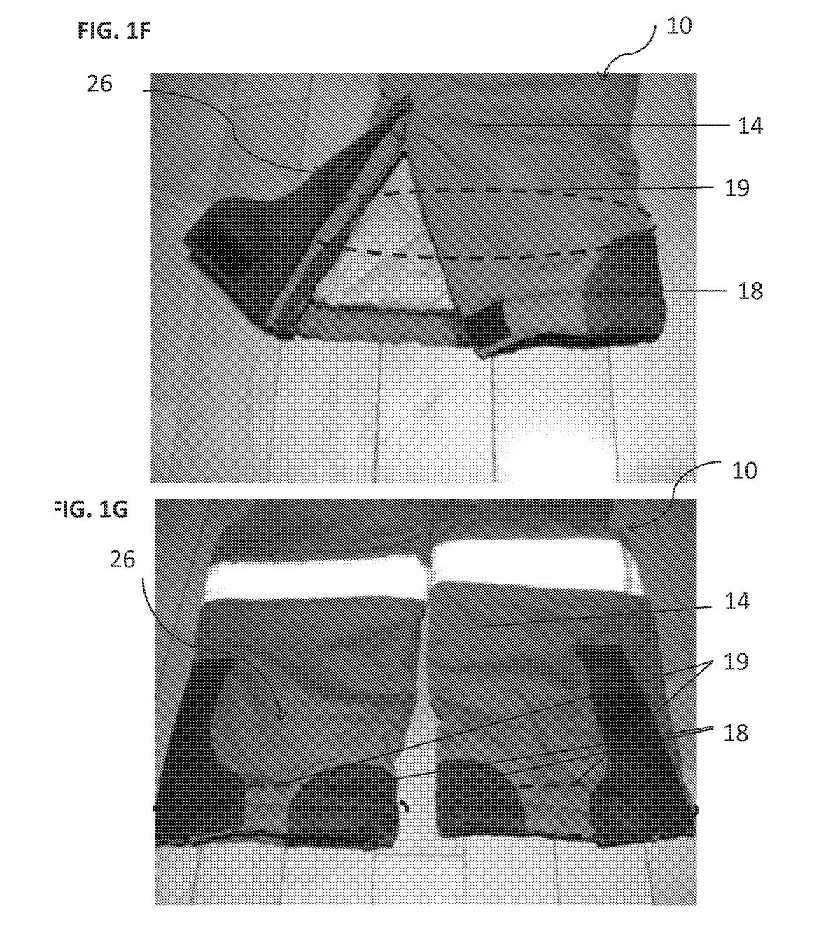

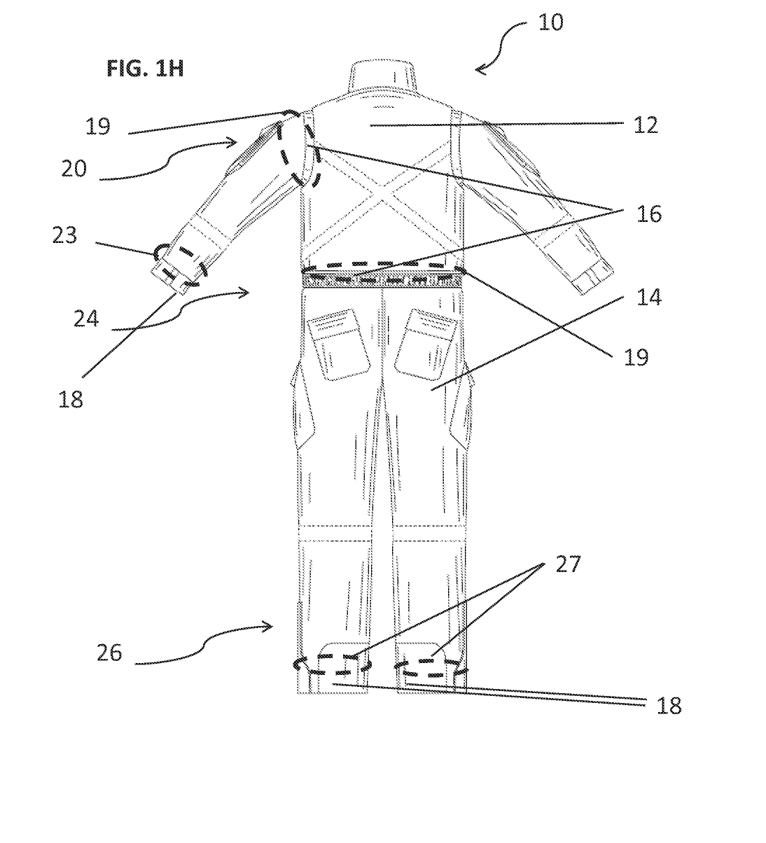

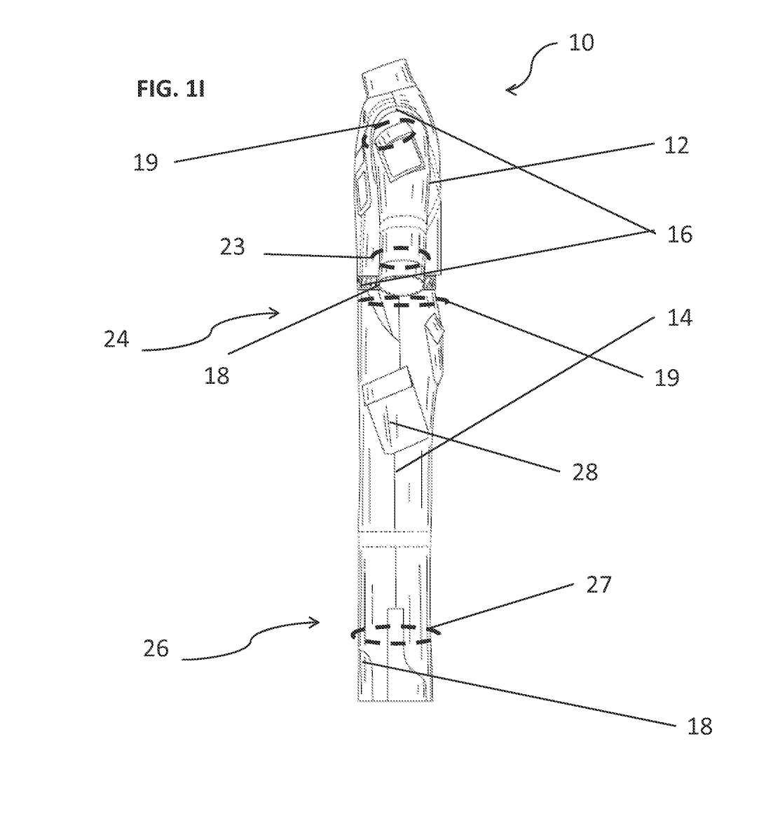

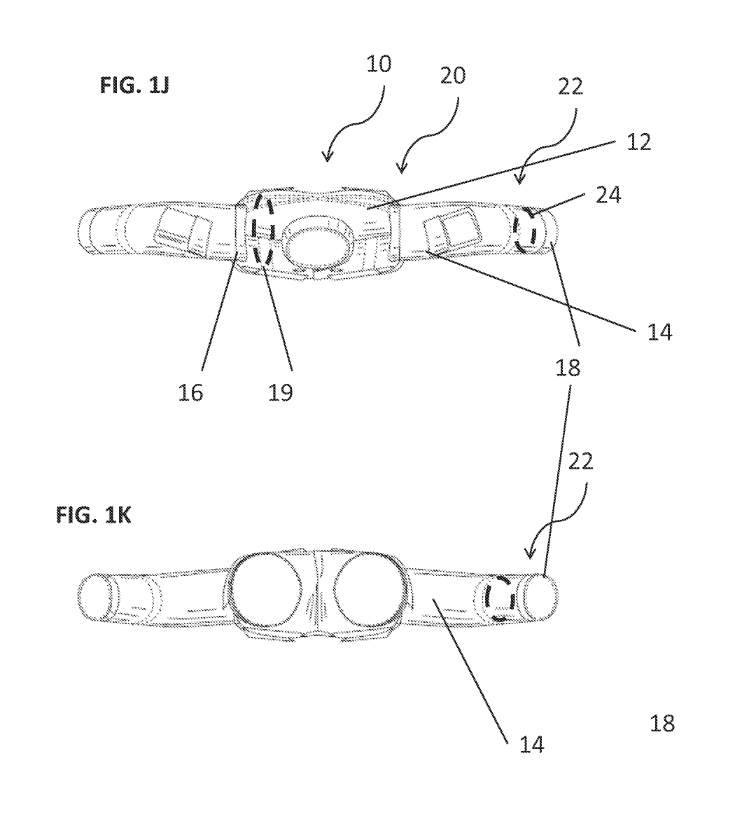

Referring now to FIGS. 1A, 1B and 1C, an exemplary safety garment 10 of the present disclosure is shown. The garment 10 may be configured to protect the user from one or more potential dangers in a hazardous environment, including but not limited to flammables, chemicals, heat, fire, oil, steam and objects, while allowing some flexibility, manoeuverability and comfort. The garment 10 comprises a covering, for example in some aspects a first covering 12 configured to cover at least part of a torso of a wearer, and a second covering 14 configured to cover at least part of a limb of the wearer. The garment 10 may further comprise additional coverings, for example in respect of hands, feet and face/head. A limb may be an arm, leg or lower extremity, for example hips and legs, of a human wearer or an animal. Safety garment 10 may further comprise a first flexible panel 16 extending from the first covering 12 to the second covering 14 and interconnecting the first covering 12 to the second covering 14. The first flexible panel 16 may completely surround a limb passage 19 in garment 10, which is adapted to receive the limb of the wearer. The limb passage 19 may be a passage or channel that enables a wearer to insert their limb therethrough into the second covering 14. Since limb passage 19 may be disposed inside garment 10, it is illustrated using a stippled line in FIGS. 1A-1G.

In some embodiments, a mechanical property of the first flexible panel 16 may be different than that of the first covering 12 and that of the second covering 14. For example, and without being limiting, the mechanical property may be elastic modulus, which is representative of the ability of flexible panel 16 to being deformed elastically (i.e., non-permanently) when a force is applied to it. In some embodiments of the present disclosure, the elastic modulus of the first flexible panel 16 is lower than that of the first covering 12 and that of the second covering 14.

The mechanical property may be flexibility, wherein the first flexible panel 16 is more flexible than the first covering 12 and the second covering 14.

The first flexible panel 16 may be disposed in various locations of safety garment 10 to facilitate movement of a limb and to accommodate relative movement between the first covering 12 and the second covering 14. For example, and without being limiting, the first flexible panel 16 may be disposed in a shoulder region 20 of the safety garment, as is shown in FIGS. 1A, 1B and 1C. The shoulder region 20 substantially corresponds to the locations on the safety garment 10 which are proximate to a wearer's shoulders when the garment is worn by the wearer. The shoulder region 20 may include region on either side a wearer's shoulder joints when wearing the safety garment 10, and may include any of the front, side and rear of the safety garment 20. Alternatively, the first flexible panel 16 may, for example, be disposed in a hip region 24 of the safety garment 10, as is shown in FIGS. 1D and 1E. The hip region 24 may substantially corresponds to a location on the safety garment 10 which are proximate to the wearer's hip when the safety garment 10 is being worn. The hip region 24 may include any of the front, sides and back of the safety garment 10. Garment 10 may further comprise at the hip region 24 an adjustable belt which may fully or partially encircle the waist of the wearer and cinch up the suit at the hips to the wearer's desired comfort. The belt may be elastic, an adjustable belt and buckle configuration, or hook and loop fabric type configuration.

Referring now to FIGS. 1F and 1G, in some embodiments, safety garment 10 may further comprise a second flexible panel 18 disposed in a second region of the safety garment, for example wrist region 22, which substantially corresponds to a location on the safety garment 10 which is proximate to a wearer's wrists when the garment 10 is worn by the wearer, or ankle region 26, which substantially corresponds to a location on the safety garment 10 which is proximate to a wearer's ankles when the garment 10 is worn by the wearer.

The second flexible panel 18 may be configured to interconnect the second covering 14 to another garment, for example a boot, helmet or a glove. The second flexible panel disposed in the second region may partially surround a passage for entry of a foot, head or hand formed between the second covering 14 and the boot, helmet or globe. The boot, helmet or glove may be made to be specifically compatible in terms of fit, colour, and materials with safety garment 10 so as to present a set of accessories for use with safety garment 10.

The garment 10 may be manufactured in a variety of different sizes and configurations suitable for users of various sizes, shapes and needs. Garment may comprise, for example, a shirt, sweater, turtleneck, trousers, jacket, coat. First covering 12 may, for example, resemble a vest or alternatively may cover a larger portion of the wearer's body. Materials suitable for first covering 12 and second covering 14 include, but are not limited to, conventional materials typical of casual, technical or other clothing or garments, for example cottons, polyesters, synthetic or natural materials or fabrics. Coverings may also be protective coverings comprised of protective materials, for example Kevlar™, Indura UltraSoft™, Nomex™, Gore-Tex™ and 3M Thinsulate™ Quilt Lining, as well as any material suitable for the intended use of the garment 10 by the wearer. Materials may preferably be flame-resistant, and may either be inherently flame resistant or be treated. Inherently flame resistant fibers are materials that have flame resistance built into their chemical structures, for example Kevlar™ and Nomex. Treated materials are made flame-resistant by the application of flame-retardant chemicals. A chemical additive in the fiber or treatment on the fabric is used to provide some level of flame retardancy. Alternatively, covering may be made of material that is waterproof, windproof, vapor or hot steam proof, or heat proof, or a material that has all these characteristics. In addition, covering may further comprise one or more reflectors, lights, or other visibility devices or markings to increase visibility of the wearer in certain circumstances.

In some embodiments, first covering 12 and/or second covering 14 may comprise a trilaminate including: an internal insulating layer, an intermediate vapour barrier and an external flame and water resistant layer. In some embodiments, the first flexible panel 16 and/or second flexible panel 18 comprise an elastomer fabric. Elastomer fabrics, for example, are those fabrics that possess the capability of elongation and yet may recover substantially fully and rapidly from elongations. In other aspects, first flexible panel 16 and second flexible panel 18 may, for example, be spandex or Lycra™.

External surfaces of safety garment 10 may also have reflective tape distributed thereon. Various linings may also be used in conjunction with first covering 12 and second covering 14, for example insulating lining, comfort lining, mesh lining, heat diffusing liners, moisture wicking liners, or any other lining suitable for the intended purposes of the garment 10 by the wearer or as desired by the wearer.

In some embodiments, first flexible panel 16 and/or second flexible panel 18 may comprise a 4-way stretch fabric. 4-way stretch fabrics may be characterised in that a given portion of fabric is able to be stretched lengthwise and widthwise while substantially returning to its original size after stretch.

Safety garment 10 may have one or more pockets 28 of varying sizes and shapes distributed on or within first covering 12, second covering 14 or disposed on or in an optional liner, and configured to carry a variety of tools, materials or any other such objects the user wishes. Pockets 28 may be constructed from any suitable material, including and not limited to a mesh material, or, for example, the same material comprising first covering 12 and second covering 14. Mesh material enabling the detection of gases therethrough utilizing a gas detection device may also be preferred. Pockets 28 may also secure objects stored within them, for example, and without being limiting, with a flap secured, for example, with a button, snap, Velcro™ or other fastening instrument. Pockets 28 may also be ergonomically angled to provide better accessibility by the user.

Safety garment 10 may also be secured on the user to allow the user to easily put on or remove safety garment 10, and to provide a secure fit, using a fastening device that is releaseably securable, for example and without being limiting, a snap, buttons, zipper, Velcro™ or combinations of these.

In operation, safety garment 10 comprising first flexible panel 16 may facilitate movement of the limb of the wearer relative to the torso of the wearer by accommodating relative movement between the first covering 12 and the second covering 14, when the safety garment 10 is being worn by a wearer. First flexible panel 16 and/or second flexible panel 18 may enable flexibility, fit and movement for the wearer while maintaining coverage and protection for the wearer, from, for example, high heat or hot steam projected towards the wearer. When exposed to potentially dangerous environmental conditions, for example high-heat steam or fire, safety garment 10 may be able to provide some coverage to the wearer's potentially vulnerable areas, for example shoulder region 20, hip region 24, wrist region 22 and ankle region 26. Safety garment 10 may maintain a close fit to wearer's body despite some of the wearer's movement or shape, and enable the first and second coverings 12, 14, preferably being flame-resistant, to protect wearer to some degree from the high heat. In the event that wearer is being subjected to hot steam, close fitment of the safety garment 10 to the wearer via first flexibility and second flexibility panels 16, 18 will, to some degree, inhibit steam from entering into or getting through safety garment 10 and causing damage or injury to the wearer.

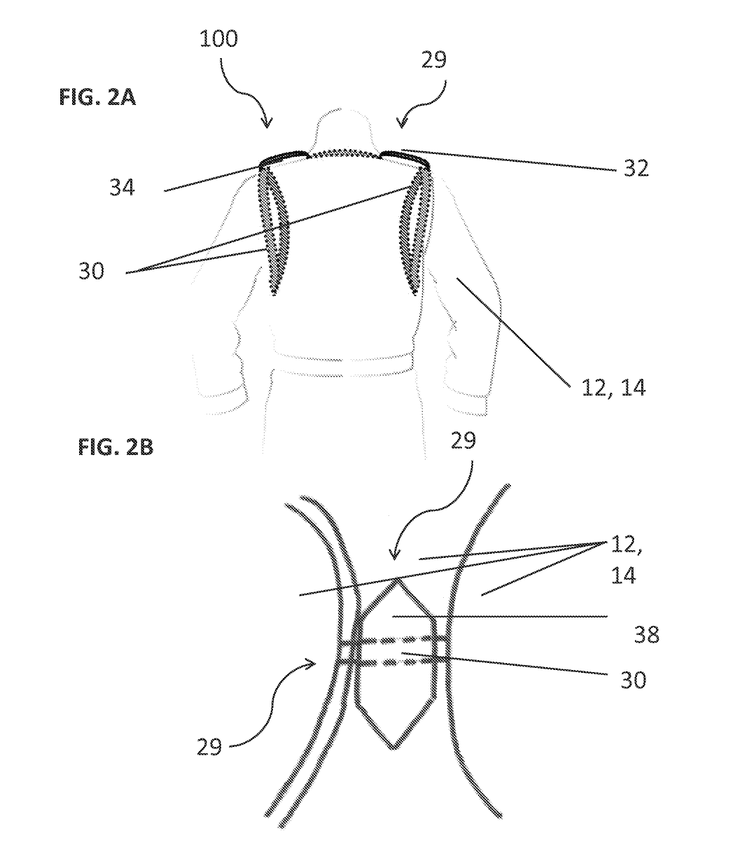

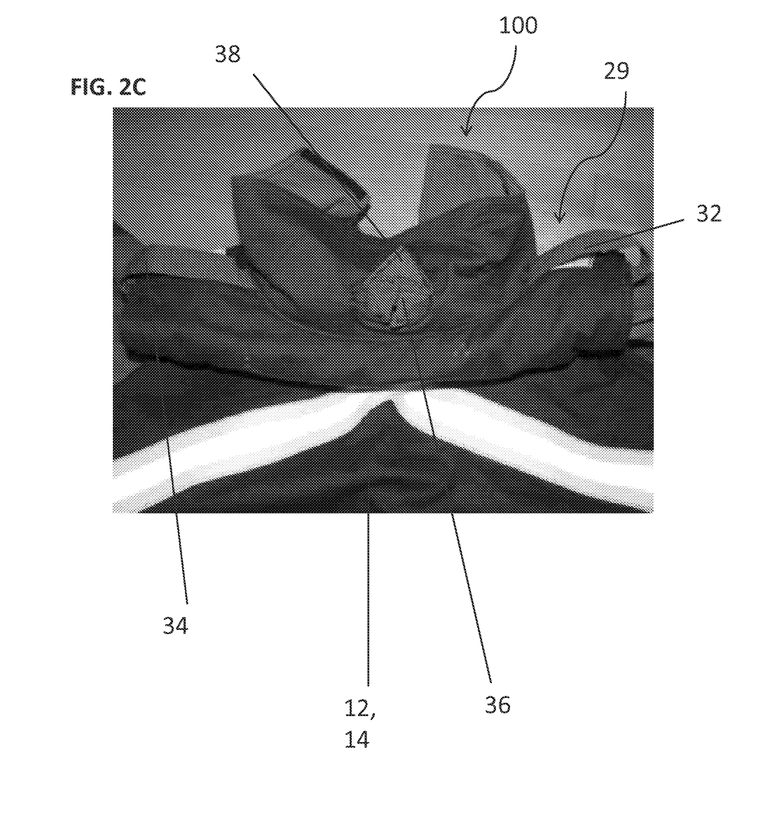

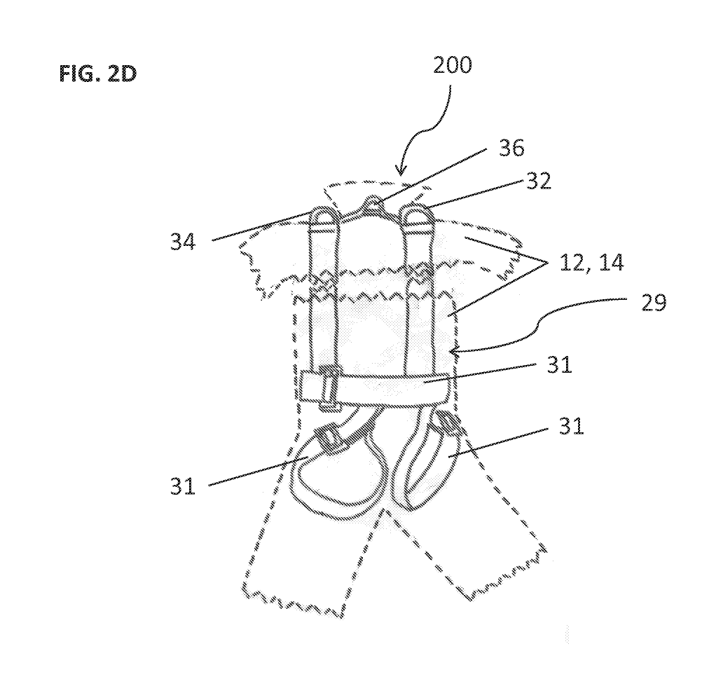

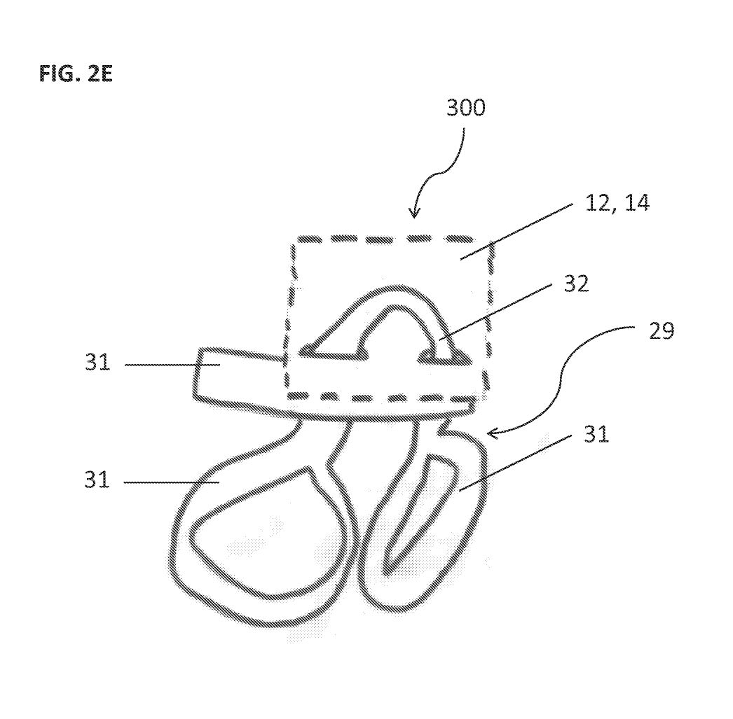

Referring now to FIGS. 2A, 2B and 2C, a safety garment 100 according to another embodiment is shown. Safety garment 100 may possess one or more of the elements described above in respect of safety garment 10. Safety garment 100 may further comprise a wearer-carrying harness 29 integrated into the covering 12, 14, the wearer-carrying harness 29 may be configured to support a weight of the wearer and permit carrying of the wearer, for example, by another individual, or by a crane or pulley system. The wearer carrying harness 29 may comprise a pair of shoulder straps 30, each of the shoulder straps 30 being configured to completely encircle a respective shoulder of the wearer. The wearer carrying harness 29 may further comprise a grasping device attached to the pair of shoulder straps 30. The grasping device may comprise one or more of first carrying handle 32, second carrying handle 34 and third carrying handle 36. Each of first, second and third carrying handles 32, 34, 36 may be configured to enable the wearer of the safety garment 100 to be grasped and carried by one or more of another individual, an emergency worker, a crane or pulley system or other such device. Each of first, second and third carrying handles 32, 34, 36 may, for example, comprise a loop-style handle comprised of a material, for example a metal, plastic or fabric, or a carabineer-style fastening system. The grasping device may be accessible from outside of the covering 12,14. Shoulder straps 30 may be disposed overtop or under the covering 12,14.

Shoulder straps 30 and grasping device together define a load path to permit carrying of the wearer. In some aspects, for example when the shoulder straps 30 may be disposed under the covering 12,14, the load path defined by the shoulder straps 30 and the grasping device extends through the covering 12, 14, for example by operation of a slit or opening in covering 12, 14 through which one of grasping device or shoulder straps may run through.

Wearer carrying harness 29 may be comprised of one or more materials or pieces, and may be secured or bonded to the safety garment by stitching, adhesives or one or more other fasteners.

Grasping device may be located in one or more locations on safety garment 100. For example, in some aspects, first carrying handle 32 may be disposed adjacent a first of the shoulder straps 30, and second carrying handle 34 may be disposed adjacent a second of the shoulder straps 30. In other aspects, grasping device may comprise a third carrying handle 36, which may be disposed about midway between the pair of shoulder straps 30.

In some embodiments, safety garment 100 may further comprise a flap 38 for selectively exposing or concealing the grasping device, or one or more of first, second and third carrying handles 32, 34, 36. The flap 38 may be configured to prevent grasping device from unintentionally catching on other surfaces during normal use of safety garment 100 by a wearer, however may be easily removable in the event that carrying of the wearer is necessary. The flap 38 may be applied to safety garment 100 or to grasping device using a fastening device, for example, one or more buttons or snaps, a hook and loop fabric, or a zipper.

Wearer carrying harness 29 may be comprised of materials described above in respect of first and second coverings 12, 14.

In operation, wearer of safety garment 100 may be carried and/or moved, by one or more individuals or by a device such as a crane or pulley system, by grasping of grasping device or one or more of first, second or third carrying handles 32, 34, 36, or attachment thereto of a pulley or crane. The load path defined by the shoulder straps 30 and grasping device may permit carrying of the wearer.

Referring now to FIG. 2D and FIG. 2E, safety garments 200, 300 according to other embodiments are shown. Safety garment 200 and safety garment 300 may possess one or more of the elements described above in respect of safety garments 10, 100. Safety garment 200 and safety garment 300 may further comprise a wearer-carrying harness 29 integrated into the covering 12, 14, the wearer-carrying harness 29 being configured to support a weight of the wearer and permit carrying of the wearer, for example, by another individual, or by a crane or pulley system. The wearer carrying harness 29 comprises one or more lower limb straps 31, each of the one or more lower limb straps being configured to completely encircle a respective lower limb of the wearer. A lower limb may be a leg, waist, or hip of the wearer.

The wearer carrying harness 29 further comprises a grasping device attached to the one or more lower limb straps 31, the grasping device comprised of one or more of first carrying handle 32, second carrying handle 34 or third carrying handle 36. The grasping device may be disposed in various locations on the respective safety garment. For example, and without being limiting, grasping device may be located in substantially the same location as with respect to safety garment 100, as shown in FIG. 2D, or alternatively, as shown in FIG. 2E The one or more lower limb straps 31 may be disposed overtop or under the covering 12,14, and may be sewn to the covering 12, 14.

One or more lower limb straps 31 and grasping device together define a load path to permit carrying of the wearer. In some aspects, for example when the one or more lower limb straps 31 may be disposed under the covering 12,14, the load path defined by the one or more lower limb straps 31 and the grasping device extends through the covering 12, 14, for example by operation of a slit or opening in covering 12, 14 through which one or grasping device runs through.

Wearer carrying harness 29 may be comprised of one or more materials or pieces, and may be secured or bonded to the safety garment by stitching, adhesives or one or more other fasteners.

In operation, wearer of safety garment 200 may be carried and/or moved, by one or more individuals or by a device such as a crane or pulley system, by grasping of grasping device or one or more of first, second or third carrying handles 32, 34, 36, or attachment thereto of a pulley or crane. The load path defined by the one or more lower limb straps 31 and grasping device may permit carrying of the wearer.

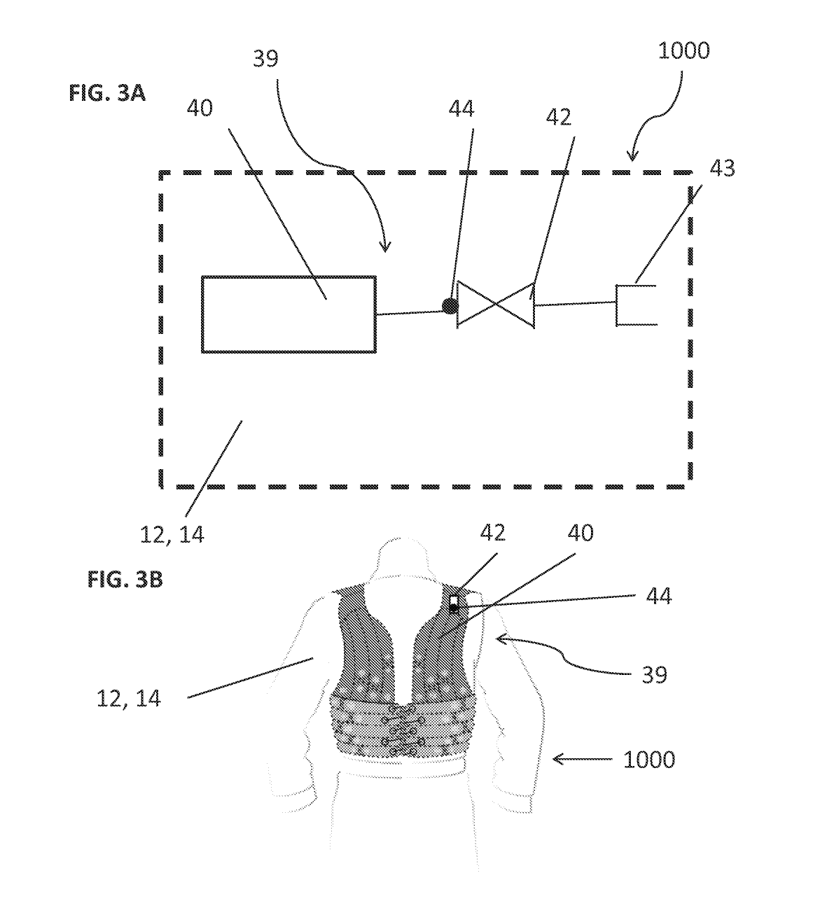

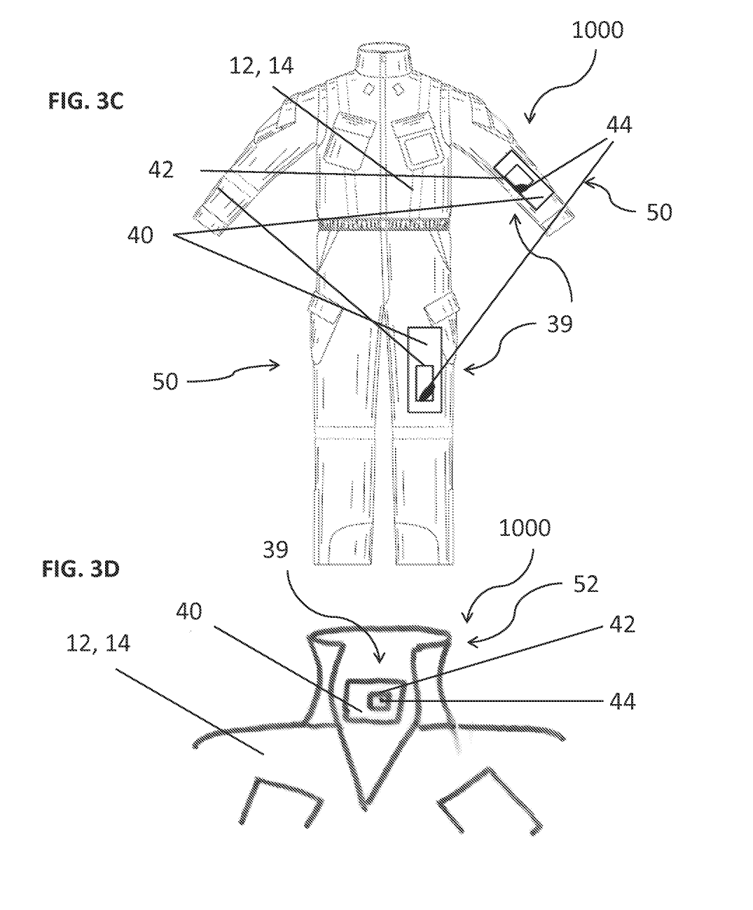

Referring now to FIG. 3A, a schematic view of a safety garment according to another aspect is shown. Safety garment 1000 may comprise one or more of the elements described above in respect of safety garment 10, safety garment 100, safety garment 200 and safety garment 300. Safety garment 1000 may further comprise a refillable heat extraction pack 39 attached to the covering 12, 14, where the pack 39 defines a chamber 40 for receiving and releasably retaining a cooling medium 46 therein. The refillable heat extraction pack 39 may, for example, be under covering 12,14, or on its external surface, however placement under covering 12,14 may be preferred.

The chamber 40 may be in a variety of shapes and sizes, depending on its location on safety garment 1000. The chamber 40 may be comprised of a flame resistant material. The chamber 40 may, for example, comprise a vest-like shape or configuration, as may be shown in FIG. 3B.

Refillable heat extraction pack 39 may comprise a flow control device 42 for permitting the release of the cooling medium 46 from the chamber 40 via port 44 after a cooling capacity of the cooling medium 46 has been reduced. Flow control device 42 may operate to allow the chamber 40 to be filled, emptied and/or refilled with cooling medium 46 via port 44. Flow control device 42 may comprise a valve for allowing cooling medium 46 into the chamber 40 into or out of chamber 40. In some aspects, a quick-connect interface 43 may be provided to enable quick and easy filling and/or refilling of chamber 40 with coolant medium 46.

Heat extraction pack 39 may be removeably attached to the covering 12, 14 using one of a hook and loop fabric, a zipper and a button or snap.

Heat extraction pack 39 may be disposed in one of a torso portion 48 of covering 12, 14, which may be proximate to the torso of the wearer of safety garment 1000, a limb portion 50 of covering 12, 14, which may be proximate to a limb of the wearer, for example an arm or leg, or a collar portion 52 of the covering 12,14, which is proximate to the neck of the wearer of safety garment 1000.

The heat extraction pack 39, including for example chamber 40, may comprise any suitable material and may preferably be a flame resistant material.

Cooling medium 46 may be any substance that would function to extract heat from a heat source, for example a wearer of garment 1000. For example, cooling medium may be one of liquid CO₂, gaseous CO₂, dry ice and liquid nitrogen.

In operation, heat extraction pack 39 defining chamber 40 may be filled or refilled with cooling medium 46 via flow control device 42, either while removably attached to safety garment 1000 via covering 12, 14, or alternatively, while removed from covering 12, 14. Heat extraction pack 39, if not already, may then be attached to covering 12, 14. Proximity of the heat extraction pack 39 to the wearer of safety garment 1000 may enable absorption of heat of the wearer of safety garment 1000 by the heat extraction pack 39, cooling the wearer. Absorption of heat from specific areas may be achieved through selective placement of heat extraction pack 39 at torso portion 46, at limb portion 50 and/or at collar portion 52 by the wearer. Once the cooling capacity of the cooling medium 46 is reduced, cooling medium 46 may be released from chamber 40 via port 44 using flow control device 42, for example, for disposal or cooling for reuse. If so desired, heat extraction pack 39 may be refilled with cooling medium 46.

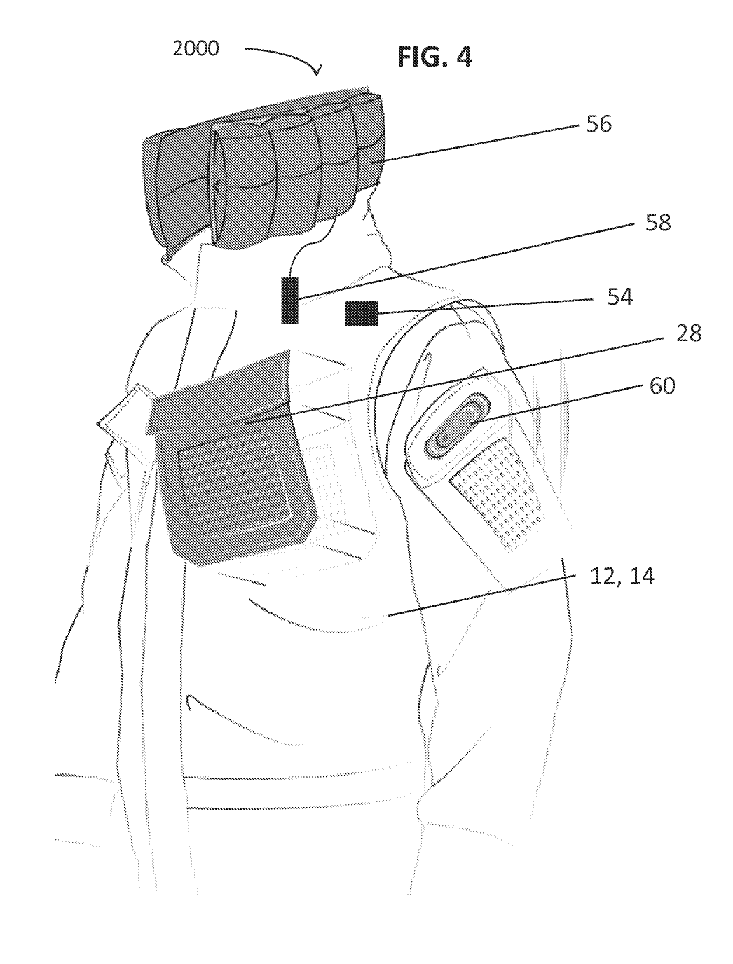

Referring now to FIG. 4, shown is a safety garment 2000 according to another aspect of the present disclosure. Safety garment 2000 may also possess one or more of the elements described above in respect of safety garment 10, safety garment 100, safety garment 200, safety garment 300 and/or safety garment 1000. Safety garment 2000 may further comprise a sensor 54 for detecting an event associated with the wearer. For example, and without being limiting, sensor 54 may be one or more sensors for detecting the wearer's identity, a thermometer for detecting user and/or ambient temperature, an accelerometer for detecting acceleration, for example to detect a user falling or tripping, a smoke detector for detecting the presence of smoke, a gas detector for detecting and/or identifying certain gasses in the environment, for example carbon monoxide, a light sensor for detecting the presence or quality of light conditions, a moisture sensor for detecting ambient moisture levels, a sensor for detecting air quality, or biosensors for detecting various biometric data of the user including but not limited to heart rate, blood pressure, or blood oxygenation. Sensor 54 may be located in various locations on safety garment 2000, distributed in multiple locations, or attached to the wearer directly. Further, sensor 54 may be embedded in safety garment 2000 so as to be substantially waterproof and/or flame-resistant, enabling functionality in a variety of environmental conditions.

The event detected by sensor 54 may include any event for which a wearer may be at increased risk of injury or danger, but may also include one or more of sudden increase in ambient temperature, a sudden decrease in ambient temperature, a sudden increase in ambient moisture level, a sudden acceleration of the garment 2000, a sudden decrease in blood oxygenation of the wearer, a sudden decrease in blood pressure of the wearer, a sudden decrease in pulse rate of the wearer, the detection of noxious gases in the ambient air including carbon monoxide, the wearer departing from a pre-determined path, or a sudden increase in ambient smoke. As used herein, “sudden” denotes a significant increase or decrease in a parameter within a short period of time ranging from more than 0 seconds to as high as 5 seconds.

Safety garment 2000 may comprise a deployable shield 56 attached to the covering 12, 14. Safety garment 2000 may comprise an actuator 58 operatively coupled to the sensor 54 and to the deployable shield 56, the actuator 58 being configured to cause deployment of the deployable shield 56 upon detection of the event by the sensor 54.

The deployable shield 56 may, for example, comprise an erectible or expandable shield or barrier, for example and without being limiting, a kevlar barrier, that is folded or rolled up and substantially out of view when undeployed, and is able to protect the wearer from certain events when deployed. Alternatively, deployable shield 56 may be inflatable, and configured to expand upon deployment. Deployable shield 56 may be constructed from any material described above in relation to covering 12, 14. When not inflated, deployable shield 56 may be kept in a substantially compact state in a portion of covering 12, 14, for example, to enable better sight and movement of the wearer of safety garment 2000.

In some aspects, the actuator 58 may, for example, comprise a source of compressed gas attached to the covering 12, 14. In other aspects, the deployable shield 56 is inflatable and the actuator 58 comprises a source of compressed gas that is delivered to the deployable shield 56 upon detection of the event by the sensor 54. The source of compressed gas may be a compressed gas container, or may be a container which produces the gas by a chemical reaction when activated. Alternatively, the source of compressed gas may be located remotely and attached to covering 12, 14 via a long inlet or flexible tube.

Deployable shield 56 may be configured to provide protection to one or more areas of the wearer of safety garment 2000. For example, and without being limiting, deployable shield 56 may be configured to protect the wearer's head and neck, or alternatively configured to protect the wearer's spine. Alternatively, deployable shield 56 may be configured to protect vulnerable areas of the wearer, for example, vulnerable joints (i.e., hips, knees, elbows).

Safety garment 2000 may, in some aspects, comprise a communication device 60 configured for communicating the event with a remote communication device; the actuator 58 operatively coupled to the communication device 60, the actuator 58 being configured to cause deployment of the deployable shield 56 upon the communication device 60 receiving the event from the remote communication device. Communication device 60 may be a device configured to communicate, for example by sending or receiving, the event to a remote communication device. Communication device 60 may send the event detected by sensor 54 to a remote communication device, perhaps notifying off-site personnel of specific environmental conditions or of the need for emergency help for the wearer in case of a sudden increase in ambient temperature, perhaps signalling a fire, or a trip or fall of the user, or the user departing from an expected course or path typical of their duties. Communication device 60 may, without being limiting, be a Wi-Fi transmitter, a Radio Frequency (RF) chip, a GPS chip, a cellular radio, or any other suitable communication device. Further, communication device 60 may also function as a keyfob for access to restricted areas.

In operation of garment 2000, sensor 54 may detect an event, for example, a sudden increase in ambient moisture and/or temperature due to a significant steam leak proximate to safety garment 2000. Upon detection of the event by the sensor 54, the actuator 58 may, for example, deliver compressed gas to the deployable shield 56, and deploy the deployable shield 56 by inflation, which may provide protection to the head and neck of the wearer of safety garment 2000 by deflecting, blocking or shielding a portion of the steam, thereby potentially minimizing the risk of the steam injuring the wearer.

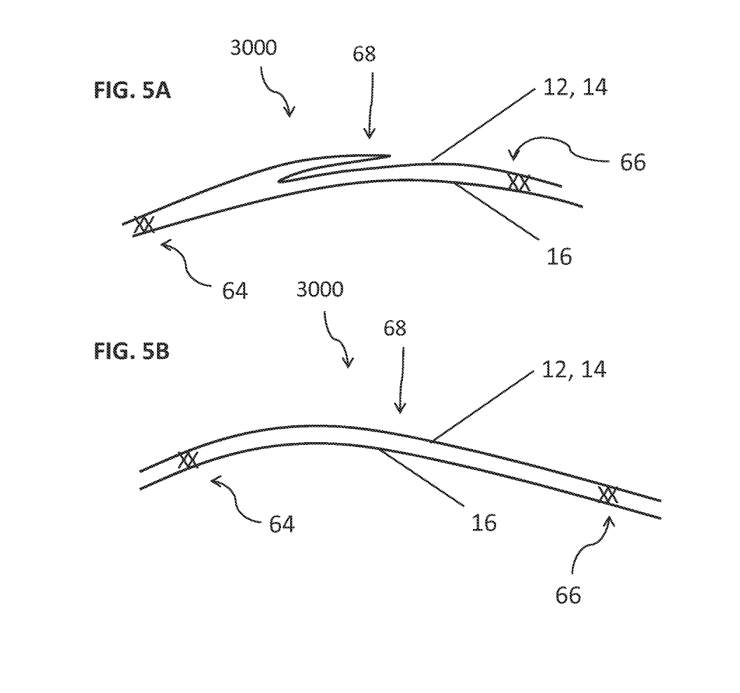

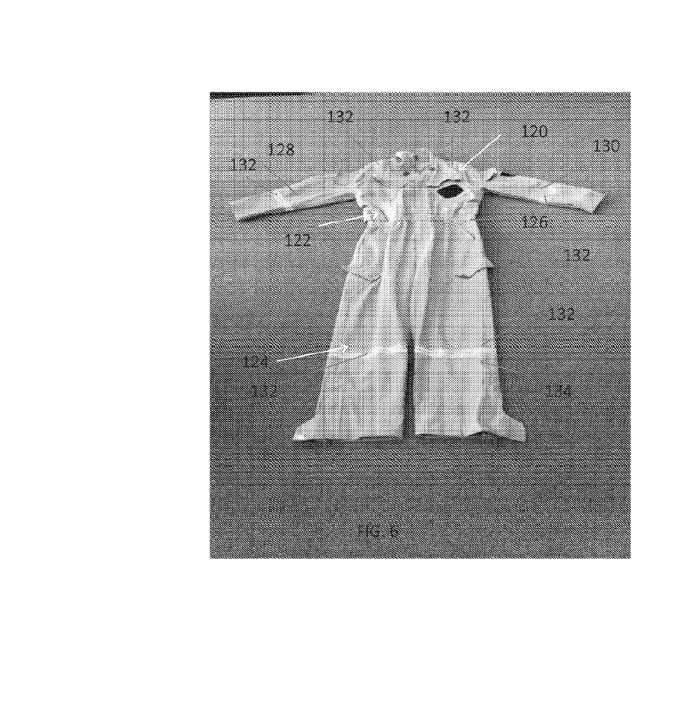

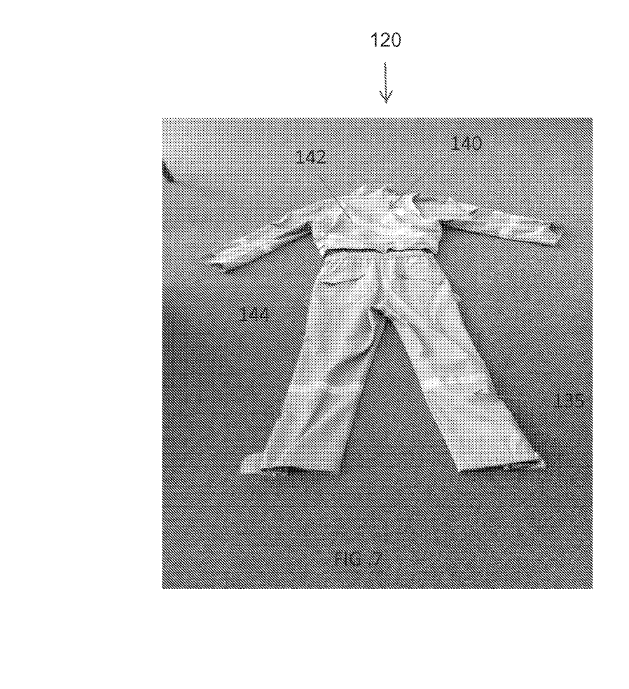

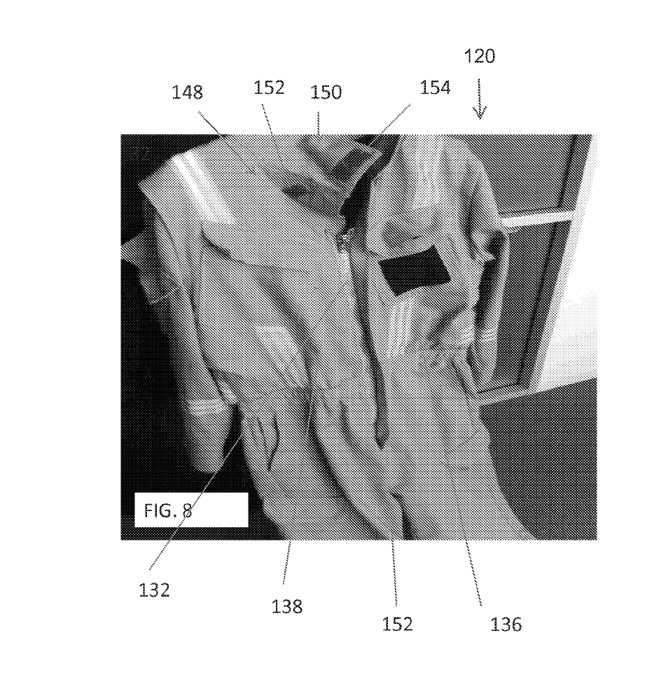

Referring now to FIGS. 5A and B, there is shown exemplary garment 3000 according to another aspect of the present disclosure. Garment 3000 may also possess one or more of the elements described above in respect of garment 10, garment 100, garment 200, garment 300, garment 1000 and/or garment 2000. Garment 3000 may comprise a covering 12, 14 configured to cover at least a portion of a wearer, the covering 12, 14 extending between and interconnecting a first location 64 and a second location 66 of the garment 3000, the second location 66 being spaced apart from the first location 64. The garment 3000 may comprise a flexible panel 16, the covering 12, 14 overlaying the flexible panel 16 and being attached to the covering at the first location 64 and the second location 64. The flexible panel 16 may, for example, comprise a liner covering at least a part of the torso or a limb of the wearer, and may, for example comprise a liner of substantially all of the covering 12, 14.

The first flexible panel 16 may be attached to the covering 12, 14 using, for example, a bonding agent, for example a glue, or sewn to the covering 12, 14. The covering may comprise an excess material 68 between the first location 64 and the second location 66. The excess material 68 may, for example, be folded or rolled, or alternatively may comprise a folded knife-style pleat, or folded pleat. The excess material 68 may be configured to accommodate a stretching of the flexible panel 16 in a first direction, for example, when the wearer of safety garment 3000 moves, bends, sits, or proceeds to conduct an activity or task. The excess material 68, for example, by partially, substantially or fully unrolling, unfolding or extending in that first direction, may accommodate the stretching of the flexible panel 16 in the first direction, as shown in FIG. 5B, which, in part, shows covering 12, 14 in an expanded, unrolled or extended state relative to FIG. 5A due to stretching of flexible panel 16. The excess material 68 may be located at one or more locations on covering 12, 14, and may be located at areas where flexibility and/or comfort of the wearer may be preferred, for example, adjacent to joints of limbs of the wearer, adjacent to the waist of the wearer, or elsewhere.

In operation of garment 3000, wearer of garment 3000 will move or conduct any activity. In doing so, flexible panel 16 may stretch in a direction, extending or lengthening in that direction. The excess material 68 may extend, expand, unfold or unroll in that direction, as the case may be, accommodating the increased length, elongation or stretching of the flexible panel 16, and thereby facilitating and/or accommodating movement of the wearer. This may provide the wearer with flexibility or comfort when using garment 3000.

Aspects of garments 10, 100, 200, 300 1000, 2000 and 3000 may be equally applicable to a non-safety garment, for example conventional or casual clothing such as shirts, jackets, coats or sweaters. For example, a garment may possess one or more of the elements described above in respect of safety garment 10, safety garment 100, safety garment 200, safety garment 300, safety garment 1000, safety garment 2000 and/or safety garment 3000, however, may comprise conventional fabrics and materials, and may be suitable in a wider range of applications. Alternatively, aspects of safety garments 100, 200, 300 1000, 2000 or 3000 may be incorporated into conventional shirts, jackets, coats, sweaters, trousers or pants.

Safety garments 10, 100, 200, 300, 1000, 2000, 3000 may be adapted and configured for use with animals, for example canines, who may be exposed to dangerous or hazardous environments. For example, canine units used for police or tactical operations, explosives detection, or those used in rescue operations, would benefit as wearers of any one of safety garments 10, 100, 200, 300, 1000, 2000, 3000. Certain modifications for safety garments 10, 100, 200, 300, 1000, 2000, 3000 may be necessary for use with animals, for example sizing of coverings 12, 14, size and configuration of first and second flexible panels 16, 18, size and configuration of wearer-carrying harness 29, size and configuration of refillable heat pack 39, and size and configuration of deployable shield 56.

### Additional Embodiments

The additional embodiments described below and/or any features thereof may be combined or used in conjunction with the features described above in relation to safety garments 10, 100, 200, 300, 1000, 2000, 3000.

Referring to FIGS. 6 and 7, the present disclosure relates to a protective garment that may be in a form of a protective one-piece garment for covering the torso, the arms, and the legs of the user. The protective one-piece garment is preferably made of material that is waterproof, windproof, vapor or hot steam proof, or heat proof, or a material that has all these characteristics. The garment 120 includes a torso portion 22 for covering the user's torso and a pair of pants 124 for covering the user's legs. A waistband 26 delineates the torso portion 122 and the pants portion 124. A pair of arm sleeves 128 and 130 is attached to the torso portion 122 for covering the user's arms. The arm sleeves 128 and 130 are preferably long sleeves to cover the entire arm of the user. However, if desired, they may be short sleeves. The arm sleeves 128, 130 may be stretchable. The torso portion 122 includes several reflectors 132 positioned at several locations, such as on the arm sleeves 128 and 130, and the shin portions 134 of the pants. The reflector on the shin portions 134 extends all the way to the calf portions 135. At the back of the torso portion 140, diagonal reflectors 142 and 144 that cross each other are provided. The reflectors 132, 142, and 144 reflect light to allow the garment to be visible, especially at night. The torso portion 22 is stretchable relative to the bottom portion/leg portion 124.

Referring to FIG. 8, reflectors 132 are preferably also positioned on the left 36 and right sides 38 of the torso. The left side 136 and right side 138 are separated by a front zipper 146. The front zipper 146 can be unzipped to provide access and allow the user to wear the garment 120. The front zipper 146 starts from the neck area 148 past the waistband 150 and ends adjacent to the crotch area 152. The neck area 148 includes a collar 150. A pair of fasteners 152 and 154, such as hook and loop fasteners, is provided under the collar 150 to hold the collar in place when the collar is in a folded position.

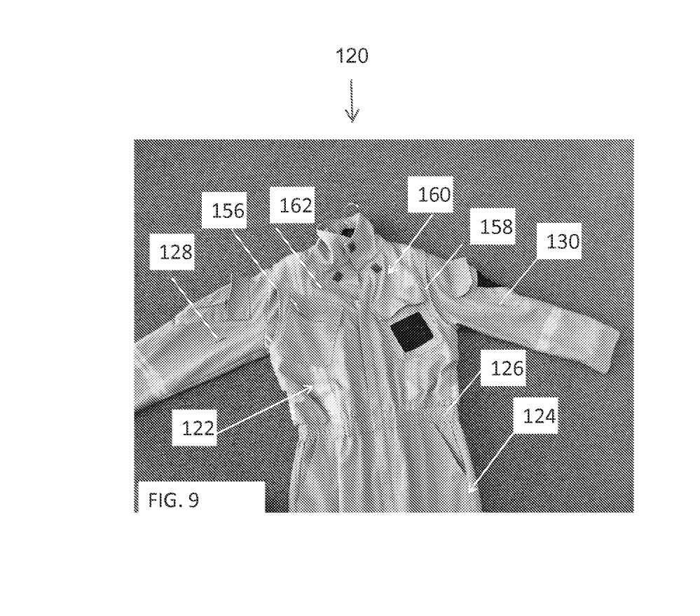

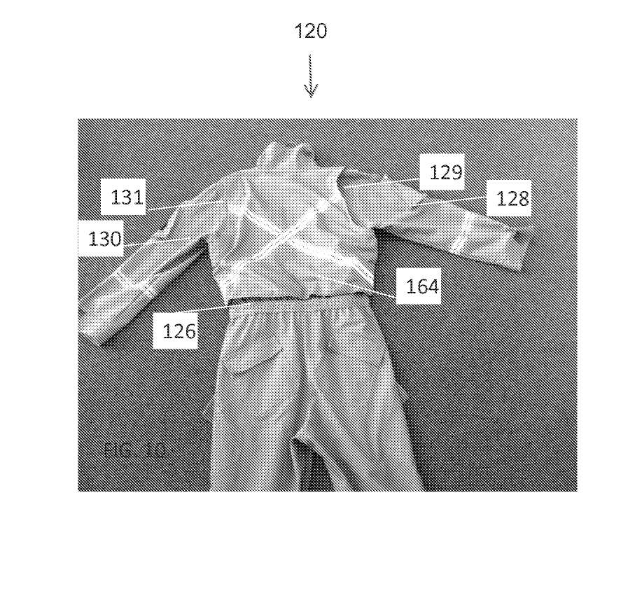

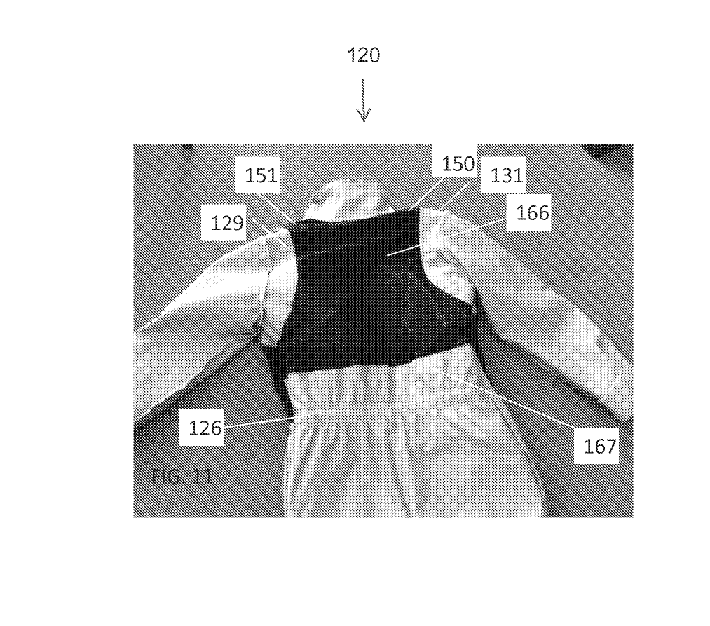

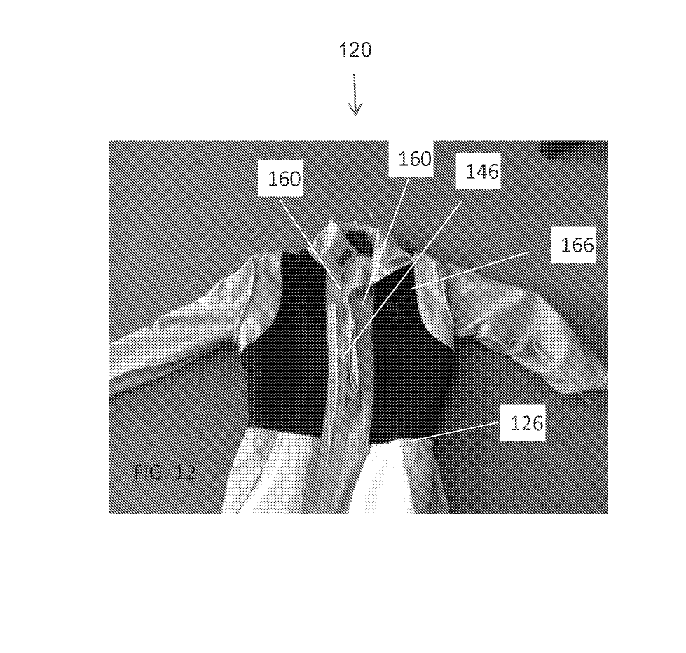

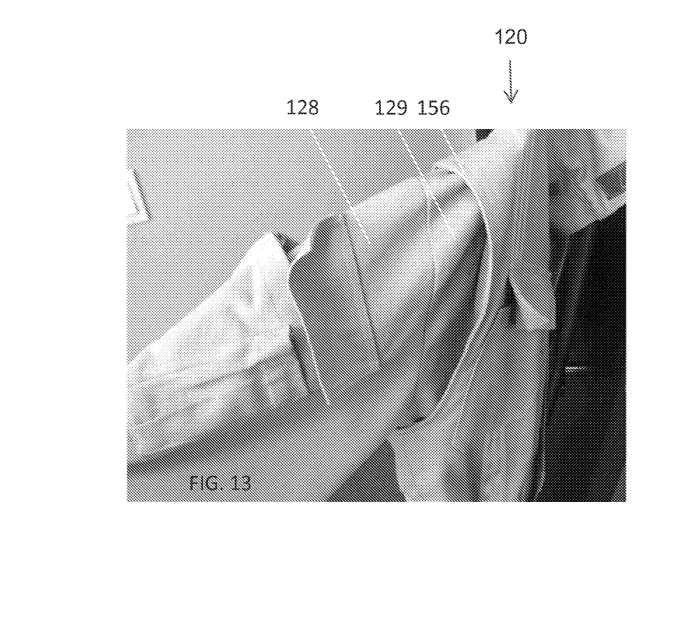

Referring to FIGS. 9-10, the torso portion 122 includes an outer layer 160 has arm cutouts 156 and 158 at the area where the arms extend from the torso. Each sleeve 128 and 130 include a circular folded knife-style pleat 129 and 131, respectively. The circular folded knife-style pleat 129 and 131 insert through their respective arm cutouts 156 and 158. The front of the outer layer 162 is connected to a waistband 26 where the torso portion 122 ends and the pants portion 124 begins. The back of the outer layer 164 is not directly attached to the waistband 126 to provide flexibility to the garment 120, which is especially beneficial when the user bends over or leans forward.

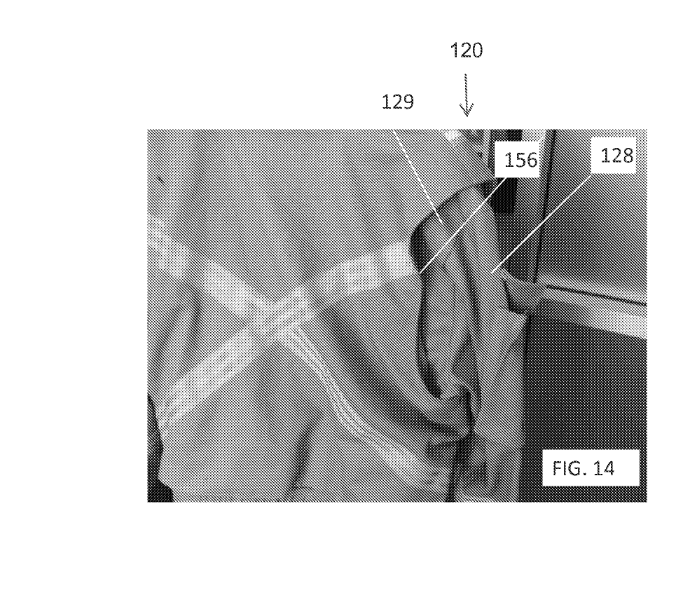

Referring to FIGS. 11-12, the torso portion 122 includes an inner layer 166 positioned inside the garment 120. The inner layer 166 may be made of mesh material, or a combination of the mesh material and the same material as the outer layer 160. The inner layer 166 is preferably made of a 4-way stretch fabric so it can stretch in vertical and horizontal directions. The inner layer 166 attaches to the outer layer 160 around the neck area 150 at the highest point of the shoulder 151, the circular folded knife-style pleats 129 and 131, and the waistband 126. The circular folded knife-style pleats 129 and 131 are created by folding the material back upon itself and sewing the folded material preferably by fire resistant thread. Each of the circular folded knife-style pleats 129 and 131 starts at the highest point of the armhole and continues down the front chest of the coverall, under the armpit and back up the backside of the garment to connect with the highest point of the shoulder where the shoulder connects with the top sleeve. The circular folded knife-style pleat 129 or 131 is positioned around the arm cutout of the garment and it connects the respective sleeve 128 and 130 to the outer layer 160 of the garment 120. The circular folded knife-style pleats 129 and 131 unfold outwards as the tensile force directed along the fabric length causes the secondary panel to separate enabling the circular folded knife-style fabric pleat 129 and 131 to unfold as the wearers extend their arms in front or above them, across their chest, beside them, or behind their bodies while at the same time allowing the circular folded knife-style pleat to retract itself into its initial folded position. The stretchable mesh aids the pleats to extend 129 and 131 further and to retract into its initial folded position.

At the back of the garment 120, the inner layer portion 167 that attaches to the waistband 126 is preferably made of the same material as the outer layer 160 for durability. The inner layer portion 167 is preferably a waist fabric extension creating a single horizontally folded knife-style pleat so that such fold remains in its folded position as the wearer wears the coverall while standing in an upright, non-bent vertical position. The horizontally folded knife-style pleat unfolds as the wearer bends over at the waist towards the ground and retracts itself into its initial folded position as the wearer bends back up to his or her initial standing upright vertical position. The inner layer portion 167 attaches to the inner layer 166 that is a stretchable mesh liner internally attached to the upper fold of the single horizontally folded knife-style pleat that stretches to release the upper fold of the single horizontally folded knife-style pleat as the wearer bends over at the waist towards the ground. The inner layer 166 in the form of a mesh liner contracts and pulls the upper fold inwards and upwards as the wearer bends back up to his or her initial standing upright vertical position.

At the front of the garment 120, the inner layer 166 is preferably made entirely of the mesh material including the portion that attaches to the waistband 126. The inner layer 166 is separated by the zipper 146. The inner layer 160 attaches to the outer layer 160 the forms the left side and the right side of the garment 120.

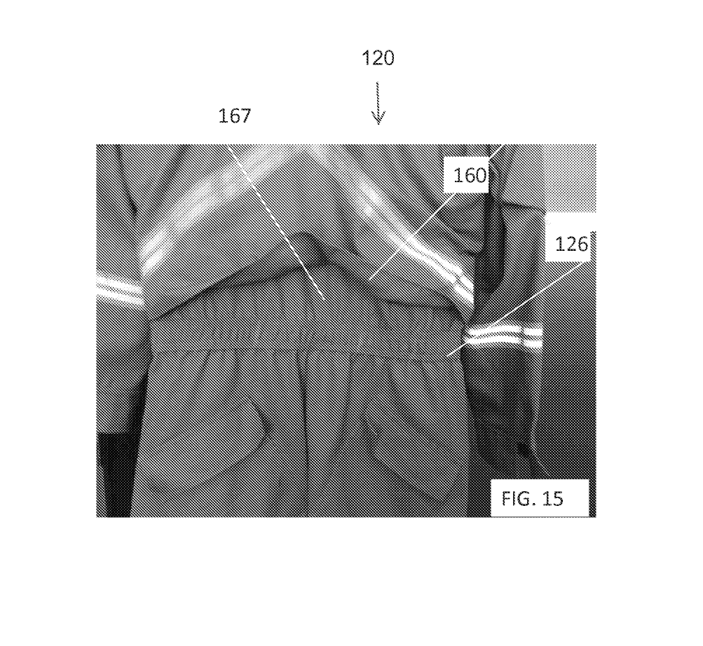

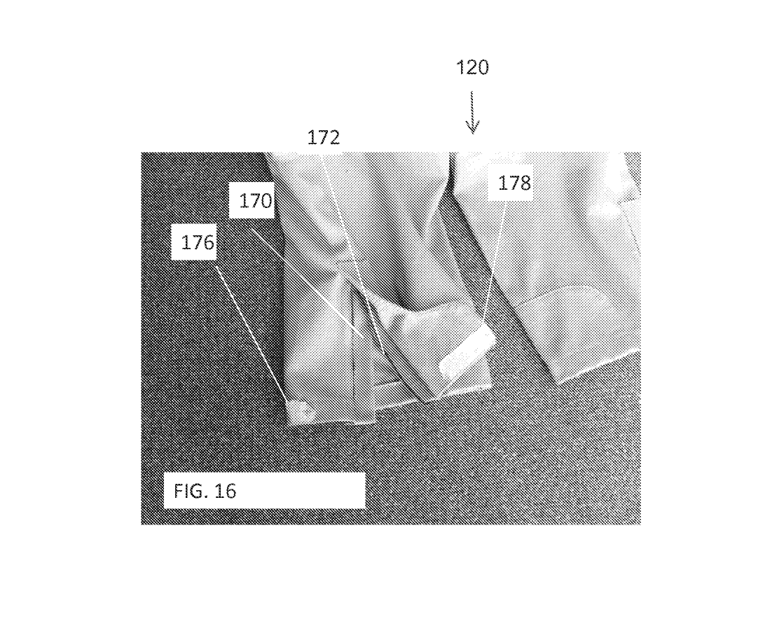

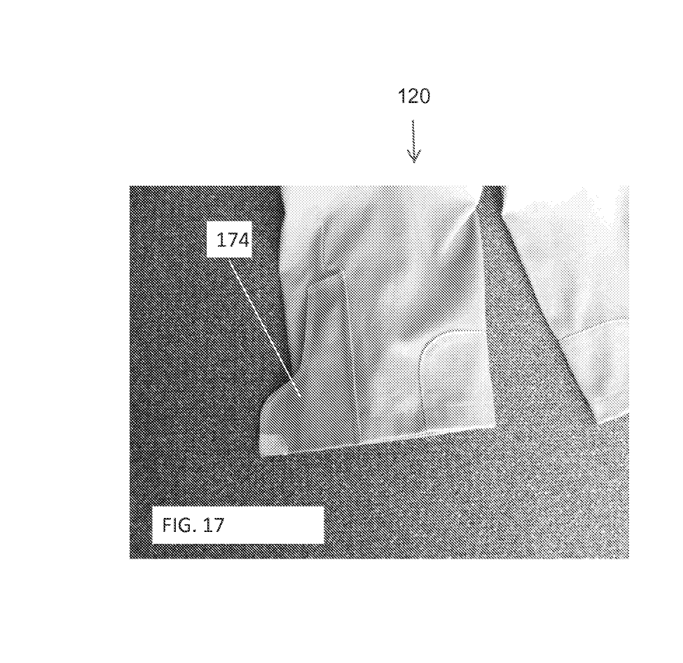

Referring to FIGS. 13-14, the sleeves 128 and 130 are inserted into their respective arm cutouts 156 and 158 of the outer layer 160 and are attached to their respective circular folded knife-style pleats 129 and 131. The circular folded knife-style pleats 129 and 131 are attached to the inner layer 166. Since they are attached to the inner layer 166 and the inner layer 166 is stretchable, the sleeves 128 and 130 are stretchable and can extend and self-contract. Referring to FIG. 15, the back of the torso portion is also stretchable due to the outer layer 160 not directly attached to the waistband 126 and the stretchable inner layer 166 being directly attached to the horizontally folded knife-style pleat 167. The horizontally folded knife-style pleat 167 is attached to the waistband 126. When the user bends over, leans forward, or leans sideways, the inner layer 166 and the horizontally folded knife-style pleat 167 stretch and cooperate with the user's movements to provide comfort to the user.

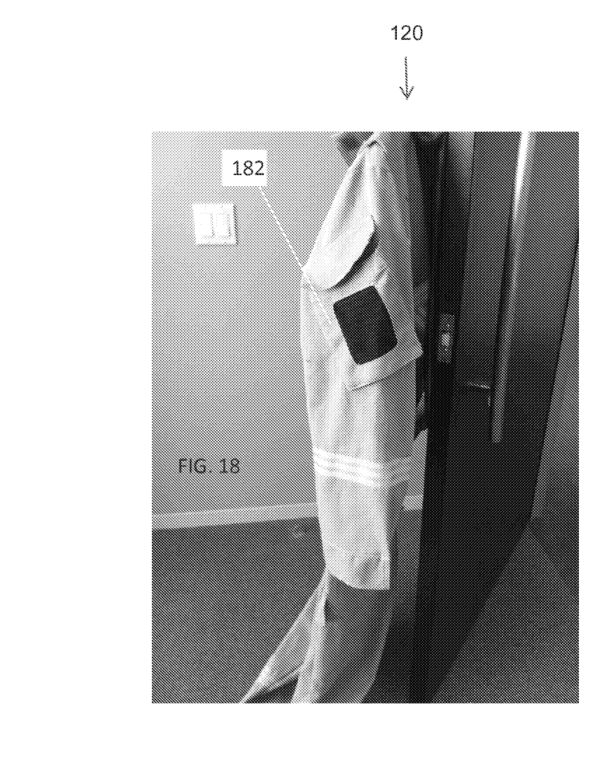

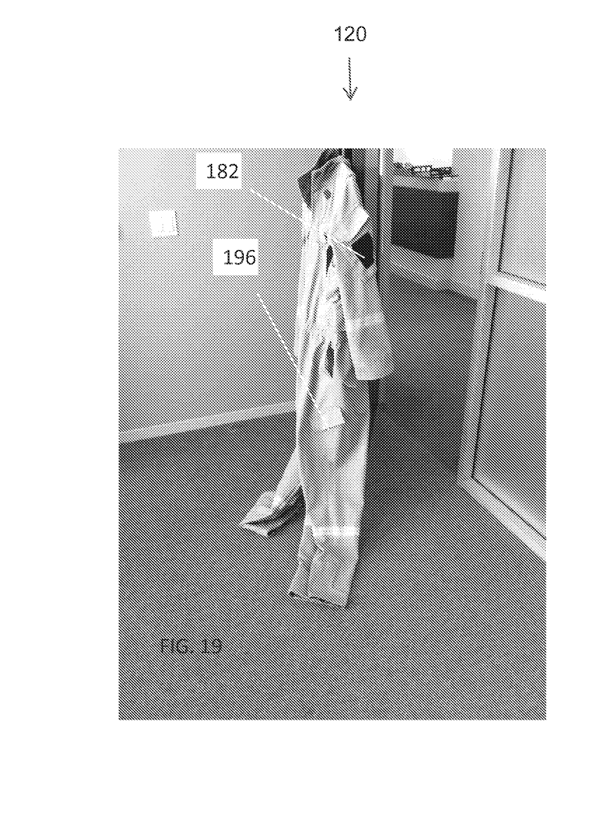

Referring to FIGS. 16-17, the garment 120 preferably has adjustable bottoms 168 to accommodate a shoe. The bottoms 168 define a slit 170 that can be opened and closed via a zipper 172. The bottoms 168 further include a flap 174 positioned over the zipper 172 for covering the zipper 172. The flap 174 has fasteners 176 and 178, such as hook and loop fasteners, for opening and closing the flap 174.

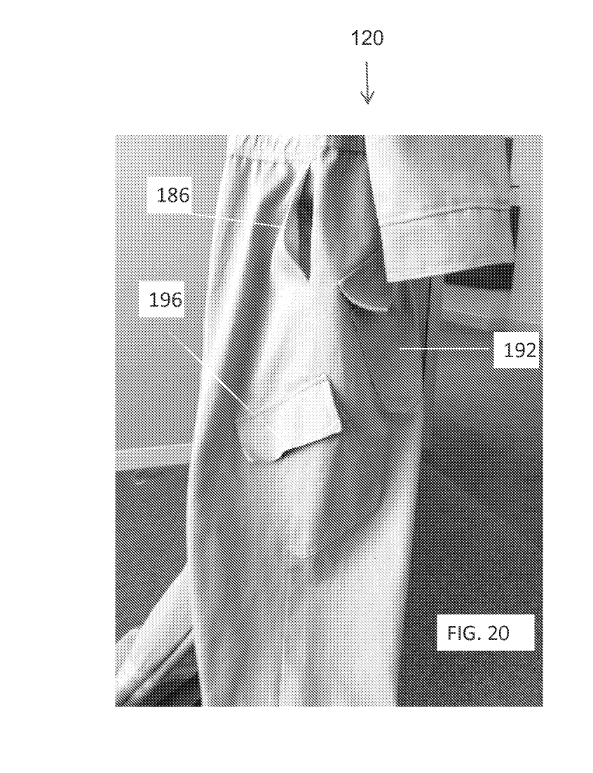

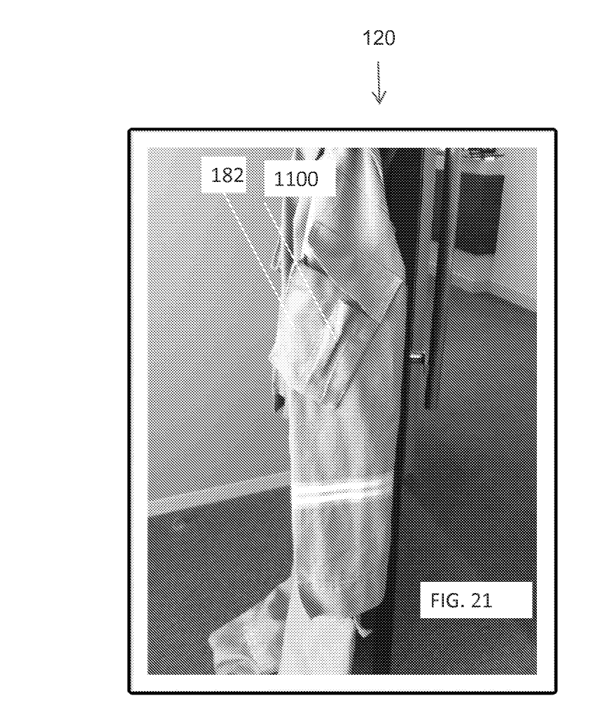

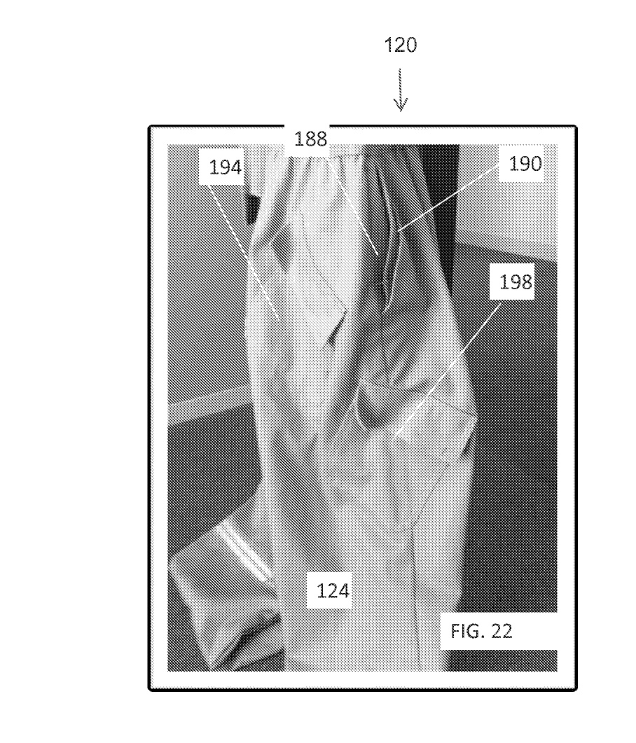

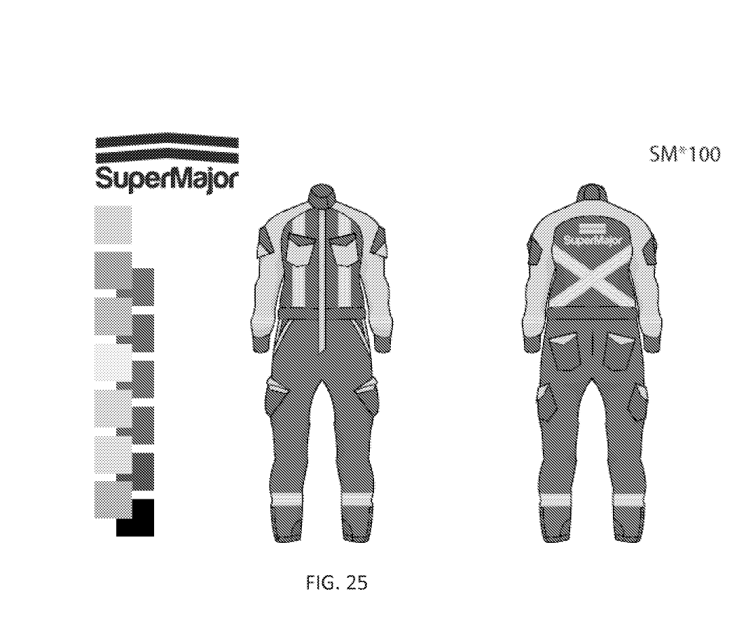

Referring to FIGS. 18-25, the garment 120 includes several ergonomically positioned pockets such that they can easily be reached. For instance, a pocket 180 is provided on each side of the sleeve 128 and 130. A pair of pockets 182 and 184 are positioned the left side and the right side, respectively, of the front of the torso portion 122. Pockets 182 and 184 are preferably angled to allow a hand to easily reach inside them. The pants 124 have pockets by the hips (pockets 186 and 188). Pockets 186 and 188 may each have a divider 190 to separate the contents. The back portion of the pants has pockets 192 and 194 that are angled towards the hips to allow a user to reach in easily. Pockets 196 and 198 are also provided on each side of the leg adjacent to the knee. Pocket 196 and 198 are also slanted to make it easy for the user to reach inside them. The pockets are preferably sized large enough to accommodate a hand, and they have flaps to cover them. The flaps have fasteners to open and close the pockets. Some of the pockets may partially include the stretchable mesh material to increase their holding capacities. The pockets may include a 4-way mesh material to accommodate a flammable gas meter electronic device. The 4-way mesh allows the ambient air to enter the pocket via the holes of the mesh so the meter can read the saturation of the flammable gas in the ambient air. Some pockets may have partitions, such as partition 1100 for elongated pieces like pens and screwdrivers.

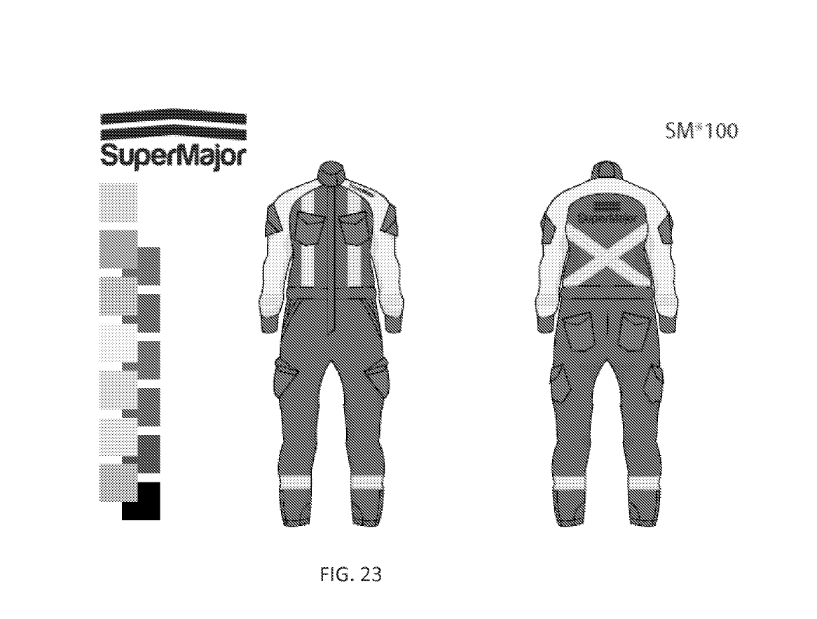

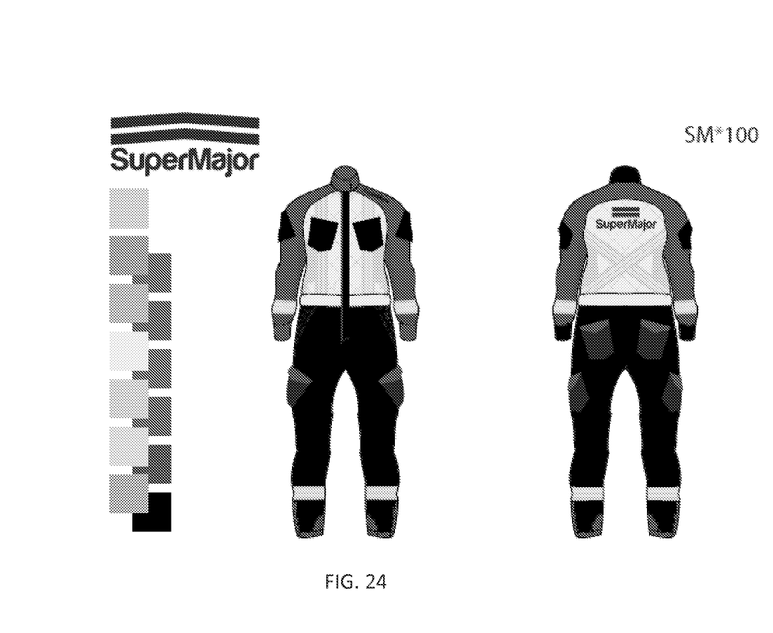

Referring to FIGS. 23-25, the garment 120 may adopt different color schemes. For instance, the reflectors may be of a first color, the pockets use a second color, the sleeves use a third color, and the rest use a fourth color. The torso portion and the pants portion may differ in color. The flaps of the pockets may differ in color with the actual pockets. The flaps of the bottom of the pants may have a color that matches the pockets and the collar. The garment may be customized with the desired color scheme.

The above description is meant to be exemplary only, and one skilled in the relevant arts will recognize that changes may be made to the embodiments described without departing from the scope of the invention disclosed. The present disclosure may be embodied in other specific forms without departing from the subject matter of the claims. Also, one skilled in the relevant arts will appreciate that while the systems, devices and assemblies disclosed and shown herein may comprise a specific number of elements/components, the systems, devices and assemblies could be modified to include additional or fewer of such elements/components. The present disclosure is also intended to cover and embrace all suitable changes in technology. Modifications which fall within the scope of the present invention will be apparent to those skilled in the art, in light of a review of this disclosure, and such modifications are intended to fall within the appended claims.

1.-71. (canceled)

72. A garment comprising:
(a) a first covering configured to cover at least part of a torso of a wearer;
(b) a second covering configured to cover at least part of a limb of the wearer; and
(c) a first flexible panel extending from the first covering to the second covering and interconnecting the first covering to the second covering, the first flexible panel completely surrounding a limb passage adapted to receive the limb of the wearer, the first flexible panel facilitating movement of the limb of the wearer relative to the torso of the wearer by accommodating relative movement between the first covering and the second covering.

73. The garment of claim 72, wherein the first covering comprises a trilaminate including: an internal insulating layer, an intermediate vapour barrier and an external flame and water resistant layer and the first flexible panel comprises an elastomer fabric.

74. The garment of claim 72, wherein the first covering comprises one of: Kevlar™, Indura UltraSoft™, Nomex™ and Gore-Tex™.

75. A safety garment for use in hazardous environments, the safety garment comprising:
(a) a covering configured to cover at least part of a wearer of the garment;
(b) a sensor for detecting an event associated with the wearer;
(c) a deployable shield attached to the covering;
(d) an actuator operatively coupled to the sensor and to the deployable shield, the actuator being configured to cause deployment of the deployable shield upon detection of the event by the sensor.

76. The safety garment claim 75, wherein the event comprises one of: a sudden increase in ambient temperature and a sudden decrease in ambient temperature.

77. The safety garment of claim 75, wherein the event comprises a sudden increase in ambient moisture level.

78. The safety garment of claim 75, wherein the event comprises one of: a sudden decrease in blood oxygenation of the wearer and a sudden decrease in blood pressure of the wearer.

79. The safety garment claim 75, wherein the event comprises a sudden increase in ambient smoke.

80. The safety garment of claim 75, wherein the sensor comprises a thermometer.

81. The safety garment of claim 75, wherein the sensor comprises a moisture sensor.

82. The safety garment of claim 75, wherein the sensor comprises a blood pressure sensor.

83. The safety garment of claim 75, wherein the sensor comprises a blood oxygenation sensor.

84. A garment comprising:
(a) a covering configured to cover at least a portion of a wearer, the covering extending between a first location and a second location spaced apart from the first location; and
(b) a flexible panel, the covering overlaying the flexible panel, the flexible panel being attached to the covering at the first location and the second location;
(c) wherein the covering comprises excess material between the first location and the second location, the excess material being configured to accommodate a stretching of the flexible panel.

85. The garment of claim 84, wherein a mechanical property of the flexible panel is different than that of the covering.

86. The garment of claim 85, wherein the mechanical property is an elastic modulus.

87. The garment of claim 86, wherein the elastic modulus of the flexible panel is lower than that of the that of the covering.

88. The garment of claim 85, wherein the mechanical property is flexibility.

89. The garment of claim 88, wherein the flexible panel is more flexible than the covering.

90. The garment of claim 84, wherein the excess material comprises a folded pleat.

91. The garment of claim 90, wherein the folded pleat accommodates the stretching by unfolding.
